(19) United States
(12) Patent Application Publication
(10) Pub. No.: US 2003/0082638 A1
(43) Pub. Date: May 1, 2003

(54) Methods for screening osteogenic compounds

(76) Inventors: Jonathan Lam, West Memphis, AR (US); F. Patrick Ross, Overland, MO (US); Steven L. Teitelbaum, St. Louis, MO (US)

Correspondence Address: Michael T. Marrah, Sonnenschein Nath & Rosenthal, Wacker Drive Station, Sears Tower, P.O. Box #061080, Chicago, IL, 60606-1080, US

(21) Appl. No.: 10/272,194

(22) Filed: Oct. 15, 2002

Related U.S. Application Data
(60) Provisional application No. 60/329,231, filed on Oct. 12, 2001.
(60) Provisional application No. 60/329,393, filed on Oct. 15, 2001.
(60) Provisional application No. 60/329,360, filed on Oct. 15, 2001.
(60) Provisional application No. 60/328,876, filed on Oct. 12, 2001.

(51) Int. Cl. G01N 33/53; G01N 33/567; A61K 38/17

(52) U.S. Cl. 435/7.2; 514/2

(57) ABSTRACT

Method of screening osteogenic compounds utilizing RANK and/or RANK pathway, compositions comprising compounds selected thereby and method for enhancing bone mass comprising administering to a subject an effective amount of said compositions.

This application is related to and claims the benefit of the following U.S. applications, which are incorporated herein by reference as if restated here in full: Serial No. 60/277,855 filed Mar. 22, 2001; Ser. No. 10/105,057 filed Mar. 22, 2002; Serial No. 60/311,163 filed Aug. 9, 2001; Ser. No. 10/215,446 filed Aug. 9, 2002; Serial No. 60/329,231 filed Oct. 12, 2001; Serial No. 60/329,393 filed Oct. 15, 2001; Serial No. 60/329,360 filed Oct. 15, 2001; Serial No. 60/328,876 filed Oct. 12, 2001; U.S. non-provisional entitled RANKL Mimics and Uses Thereof, LAM, et al., filed Oct. 15, 2002; U.S. non-provisional entitled Bone-Anti Resorptive Compounds, LAM, et al., filed Oct. 15, 2002.

This invention was made in part with Government support under National Institutes of Health Grants AR32788, AR46123 and DEO5413. The Government has certain rights in the invention.

### FIELD OF THE INVENTION

The present invention relates to methods for screening osteogenic compounds using RANK or the RANK pathway.

The present invention also relates to methods and compositions for enhancing processes of bone formation, thereby providing novel treatments for diseases or conditions which are at least partially characterized by loss of bone mass.

The present invention further encompasses compounds identified by such screening methods and compositions comprising these compounds.

The invention also relates to methods for enhancing processes of bone formation, wherein said methods comprise administering bone mass enhancing compositions provided herein.

### BACKGROUND OF THE INVENTION

Various conditions and diseases which manifest themselves in bone loss or thinning are a critical and growing health concern. It has been estimated that as many as 30 million Americans and 100 million worldwide are at risk for osteoporosis alone. Mundy et al., Science, 286: 1946-1949 (1999). Other conditions known to involve bone loss include juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia. Additionally, new bone formation is needed in many situations, e.g., to facilitate bone repair or replacement for bone fractures, bone defects, plastic surgery, dental and other implantations and in other such contexts.

Bone is a dense, specialized form of connective tissue. Bone matrix is formed by osteoblast cells located at or near the surface of existing bone matrix. Bone is resorbed (eroded) by another cell type known as the osteoclast (a type of macrophage). These cells secrete acids, which dissolve bone minerals, and hydrolases, which digest its organic components. Thus, bone formation and remodeling is a dynamic process involving an ongoing interplay between the creation and erosion activities of osteoblasts and osteoclasts. Alberts, et al., Molecular Biology of the Cell, Garland Publishing, N.Y. (3rd ed. 1994), pp. 1182-1186.

Present forms of clinically-approved bone loss therapy are primarily anti-resorptive, in that they inhibit bone resorption processes, rather than enhance bone formation. Among the agents which have been used or suggested for treatment of osteoporosis because of their claimed ability to inhibit bone resorption are estrogen, selective estrogen receptor modulators (SERMs), calcium, calcitriol, calcitonin (Sambrook, P. et al., N. Engl. J. Med. 328:1747-1753), alendronate (Saag, K. et al., N. Engl. J. Med. 339:292-299) and other bisphosphonates. Luckman et al., J. Bone Min. Res. 13,581 (1998). However, anti-resorptives fail to correct the low bone formation rate frequently involved in net bone loss, and may have undesired effects relating to their impact on the inhibition of bone resorption/remodeling or other unwanted side effects.

As a result, it would be very desirable to obtain compounds with osteogenic activity that could be used to develop therapeutics for enhancement of bone formation. Unfortunately, the number of assays currently available for screening and identifying potential osteogenic agents is very limited. One such assay is disclosed in U.S. Pat. No. 6,083,690, and it determines the osteogenic potential of a compound based on its ability to stimulate bone cells to produce bone growth factors in the bone morphogenetic family.

A key development in the field of bone cell biology is the recent discovery that RANK ligand (RANKL, also known as osteoprotegerin ligand (OPGL), TNF-related activation induced cytokine (TRANCE), and osteoclast differentiation factor (ODF)), expressed on stromal cells, osteoblasts, activated T-lymphocytes and mammary epithelium, is the unique molecule essential for differentiation of macrophages into osteoclasts. Lacey, et al., Cell 93: 165-176 (1998)(Osteoprotegerin Ligand Is a Cytokine that Regulates Osteoclast Differentiation and Activation.) The cell surface receptor for RANKL is RANK, Receptor Activator of Necrosis Factor (NF)-kappa B. RANKL is a type-2 transmembrane protein with an intracellular domain of less than about 50 amino acids, a transmembrane domain of about 21 amino acids, and an extracellular domain of about 240 to 250 amino acids. RANKL exists naturally in transmembrane and soluble forms. The deduced amino acid sequence for at least the murine, rat and human forms of RANKL and variants thereof are known. See e.g., Anderson, et al., U.S. Pat. No. 6,017,729, Boyle, U.S. Pat. No. 5,843,678, and Xu J. et al., J. Bone Min. Res. (2000/15:2178) which are incorporated herein by reference. RANKL (OPGL) has been identified as a potent inducer of bone resorption and as a positive regulator of osteoclast development. Lacey et al., supra.

In addition to its role as a factor in osteoclast differentiation and activation, RANKL has been reported to induce human dendritic cell (DC) cluster formation. Anderson et al., supra and mammary epithelium development J. Fata et al., “The osteoclast differentiation factor osteoprotegerin ligand is essential for mammary gland development,” Cell, 103:41-50 (2000). Recently, we have determined that RANKL plays a role in anabolic bone formation processes and can be utilized in methods for stimulation of osteoblast proliferation or bone nodule mineralization, as disclosed in application Ser. No. 60/277,855, filed Mar. 22, 2001 and Ser. No. 10/105,057 filed Mar. 22, 2002. In addition, this patent application discloses methods for stimulation of osteogenesis using RANK ligand fusion proteins, thereby providing novel methods of treating, preventing or inhibiting bone loss in subjects. Due to the paucity of anabolic bone agents, it would be desirable to discover or develop other compounds besides RANK ligand fusion proteins that can play a role in enhanced bone formation.

Accordingly, a need exists for novel methods of screening osteogenic compounds. Further, there is a need for novel methods and compositions that are useful in enhancing bone formation.

### SUMMARY OF THE INVENTION

Accordingly, among the objects of the invention is the provision of methods for screening for osteogenic compounds by using some portion of the RANK pathway. In addition, the present invention encompasses compounds that are identified by the screening methods disclosed herein. Further provided are methods and compositions for enhancing bone formation.

A method of the invention involves screening osteogenic compounds and includes the steps of selecting compounds that activate some portion of the RANK pathway, and performing bone formation assays with said compounds. A positive result of the bone formation assays indicates that the test compound possesses osteogenic activity. Such method of screening may also include the step of identifying compounds that led to bone formation in said assays.

Selecting compounds that bind to RANK may involve different assays, such as, e.g., binding assays wherein a compound's ability to bind to RANK is determined directly or by competition with RANKL or domains of RANKL known to bind RANK. In preferred embodiments this RANKL binding domain may consist essentially of a RANKL surface contact loop or fragment, derivative, or analog thereof. Compounds for screening may be selected from various libraries of small molecular weight compounds, peptides, or alternatively may be selected by homology modeling, computational modeling, and screening phage display libraries. RANK may be selected from a recombinant RANK protein, a RANK fusion protein, an analog, derivative or mimic thereof. In a preferred embodiment, RANK is a recombinant RANK protein.

Another method of the present invention involves screening osteogenic compounds and include the steps of incubating the desired compounds with osteoblasts, osteoblast precursors, or related cell lines and determining the activation status of said cells. The compounds to be screened include compounds selected for their ability to bind to RANK as well as those which activate and/or inactivate at least some portion of the RANK pathway regardless of RANK stimulation. Activation of osteoblasts or osteoblast precursors is determined by establishing the activation status of intracellular proteins in the incubated cells, whose activation is indicative of bone anabolism. The assays used to test for activation of intracellular proteins vary according to the protein in question and are well established in the art, e.g. Western blots, kinase and phosphatase assays. The positive outcome of the assay, i.e. a protein in its activated or deactivated state may designate the compound being tested as having the net ability to activate and/or induce proliferation of osteoblasts, osteoblast precursors or related cell lines. The compound may then be tested in a bone formation assay and identified, for example by utilizing mass spectrometry analysis.

In a preferred embodiment, the proteins that are assayed include proto-oncogene proteins, including c-Fos, and intracellular kinases, including ERK1/2, JNK, p38, PI3 kinase, Akt, and IKK. IKK functions as an upstream regulator of the transcriptional complex comprising the NFKB family. These NFKB members are well know to those versed in the art. Activation of the NFKB pathway is assessed by performing electrophoretic mobility shift assays (EMSA) on nuclear extracts. More preferably the assayed kinases are MAP kinases, including ERK1/2, JNK, and p38. Most preferably, the assayed kinase is ERK1/2.

In another preferred embodiment, the activation of intracellular proteins on the RANK pathway constitutes phosphorylation of the same. Specifically, the phosphorylated proteins include ERK1/2, PI3 kinase, Akt, JNK, and p38. Most preferably, the phosphorylated kinases are ERK1/2.

In a separate preferred embodiment, activation may be determined by measuring nuclear translocation of members of the NFKB family, using EMSA or other methods well known in the art. Nuclear NFKB may also be phosphorylated or dephosphorylated. Phosphorylation of NFKB itself, as well activity of the kinases or phosphatases involved, may also be assayed.

In another aspect, the activation of the intracellular proteins can be detected for at least about 15-30 minutes after the incubation of the compound with osteoblasts or osteoblast precursors. Preferably, the activation can be detected for 40 minutes, and more preferably it can be detected for at least 60 minutes after the interaction.

In addition, the invention provides methods of screening osteogenic compounds based on their ability to inactivate phosphatase(s) as determined from in vitro assays. Preferably, the phosphatases are MAPK specific, Akt specific, PI3 kinase specific, IKK specific, or NFKB specific. More preferably, the phosphatases are ERK1/2 specific. It is believed that additional phosphatases are likely, yet-to-be-discovered, and that the method of this invention as taught herein will apply equally to assays relating thereto. The compounds with the ability to inactivate said phosphatases can then be tested in bone formation assays to further determine their osteogenic potential.

The present invention also includes compositions for enhancing the processes of bone formation, including by stimulating bone formation. These compositions may comprise compounds identified by the screening methods disclosed herein. The invention further relates to methods for enhancing processes of bone formation, wherein said methods comprise intermittently administering to a subject an effective amount of the compositions provided herein. Thus, these methods may be used to treat diseases or conditions characterized at least in part by the loss of bone mass.

Other objects and features will be in part apparent and in part pointed out hereinafter.

### BRIEF DESCRIPTION OF FIGURES

FIG. 1 is a histological presentation of GST-RANKL stimulation of bone formation ex vivo in whole calvarial organ culture, as discussed in Example 1.

FIG. 2 is a graphic depiction of the dose-dependent increase in cavarial thickness due to GST-RANKL stimulation of bone formation in vitro, as discussed in Example 1.

FIG. 3(a) is a histological presentation of GST-RANKL stimulation of bone formation in vivo in mice, as discussed in Example 2.

FIG. 3(b) depicts a dual-energy X-ray absorptiometry (DEXA) analysis of tibial metaphyses comparing bone mineral density of animals administered GST-RANKL or control vehicle in vivo, as discussed in Example 2.

FIG. 4 is a graphic presentation of alkaline phosphatase (AP) activity following GST-RANKL exposure.

FIG. 5 is an image of a Western blot depicting the rapid activation of the members of the MAPK pathway in murine osteoclast precursors following the treatment of cells with GST-RANKL. The activation was measured at the time of GST-RANKL/RANK interaction (0 minutes) and 5, 15, and 30 minutes following the interaction. From the top, the second, fourth, and sixth panels show the total levels of JNK, p38, and ERK respectively. The first, third, and fifth panels depict the phosphorylated (activated) forms of JNK, p38, and ERK respectively.

FIG. 6 is an image of a Western blot depicting the activation of Akt in murine osteoclast precursors following the treatment of cells with GST-RANKL. The activation was monitored at the time of GST-RANKL/RANK interaction, and 5 and 15 minutes following the interaction. The bottom panel depicts the levels of total Akt at specified time points, whereas the top panel depicts the phosphorylated forms of Akt.

FIG. 7 is an image of a Western blot depicting the prolonged activation of the kinases in MAPK pathway in murine osteoblasts following the GST-RANKL treatment of cells. The time points for which the phosphorylation was measured included 0 minutes (time of GST-RANKL stimulation of cells), and 5, 10, 20, 30, and 60 minutes after exposure of cells to GST-RANKL. The kinases whose activation was measured included ERK, JNK, p38, and Akt. pERK designates phosphorylated ERK, ERK designates the total amount of the same protein, pJNK designates phosphorylated JNK, JNK designates the total amount of JNK, pp38 designates phosphorylated p38, p38 designates the total amount of p38, pAkt designates phosphorylated Akt, and Akt designates the total amount of the same protein. In the second panel, first row from the top is p-lkBα, which designates phosphorylated lkBα, whereas lkBα designates the total amount of the same protein.

FIG. 8 is an image of a Western blot depicting the prolonged activation of ERK1/2 in murine osteoblast precursors following the treatment of cells with GST-RANKL. The time points at which ERK1/2 activation was measured include 0, 5, 10, 20, 30, and 60 minutes following exposure of cells to GST-RANKL. pERK designates phosphorylated ERK whereas ERK designates the total amount of the same protein.

FIG. 9 is an image of a Western blot depicting the prolonged activation of c-Fos following treatment of cells with GST-RANKL. Time points at which activation was measured were 0, 30, 60 and 120 minutes following exposure of cells to GST-RANKL. B-actin was used as a control.

FIG. 10 is a graphic presentation of the increase in bone mineral density (BMD) due to GST-RANKL stimulation of bone formation in mice, as discussed in Example 8.

### ABBREVIATIONS AND DEFINITIONS

To facilitate understanding of the invention, a number of terms are defined below:

“RANKL” or “RANK ligand” are used interchangeably herein to indicate a ligand for RANK (Receptor Activator of NFKB).

“RANK pathway” refers to RANK and the downstream proteins relating to bone formation capable of being activated by a signal from RANK and including NFKB translocation, proto-oncogene proteins, including c-Fos, and intracellular kinases, including ERK1/2, IKK, PI3 kinase, Akt, JNK, and p38.

“MAP kinase” or “MAPK” are used interchangeably herein, and are abbreviations for mitogen activated protein kinase. The MAPK family comprises three proteins, ERK1/2, JNK, and p38.

“ERK1/2” refers to ERK1 and ERK2, which are abbreviations for extracellular signal-regulated kinase 1 and extracellular signal-regulated kinase 2, respectively.

JNK is an abbreviation for c-jun N-terminal kinase.

p38 is a kinase of 38 kDa, which is a member of the MAPK family of kinases.

Akt is Akt serine threonine kinase.

IKK is an abbreviation for IkappaB (IKB) kinase.

The terms “compound” and “molecule” are used interchangeably herein.

The terms “enhance bone mass”, “enhance bone formation”, “osteogenic potential”, “osteogenic activity” and similar terms are used interchangeably herein to refer to any compound that is able to enhance bone formation, as determined from bone formation assays, and specifically including formation resulting from anabolic activity.

The terms “activation” and “deactivation” when used in reference to biochemical actions upon molecules are meant to be inclusive of any action on that molecule the net and ultimate result of which is enhanced bone formation.

As used herein, “ERK1/2-specific phosphatase” refers to phosphatase(s), whose function is to dephosphorylate (inactivate) the active forms of ERK1/2.

As used herein, “NFKB phosphatase(s)” refers to phosphatase(s), whose function is to dephosphorylate (inactivate) the active forms of NFKB.

By the term “an effective amount” is meant an amount of the substance in question that one skilled in the art would expect to a statistically significant effect. For example, an “effective amount” for therapeutic uses is the amount of the composition comprising an active compound herein required to provide a clinically significant increase in healing rates in fracture repair; reversal or inhibition of bone loss in osteoporosis; prevention or delay of onset of osteoporosis; stimulation and/or augmentation of bone formation in fracture non-unions and distraction osteogenesis; increase and/or acceleration of bone growth into prosthetic devices; repair or prevention of dental defects; or treatment or inhibition of other bone loss conditions, diseases or defects, including but not limited to those discussed herein above. Such effective amounts will be determined using routine optimization techniques and are dependent on the particular condition to be treated, the condition of the subject, the route of administration, the formulation, and the judgment of the practitioner and other factors evident to those skilled in the art. The dosage required for the compounds of the invention (for example, in osteoporosis where an increase in bone formation is desired) is manifested as that which induces a statistically significant difference in bone mass between treatment and control groups. This difference in bone mass may be seen, for example, as at least 1-2%, or any clinically significant increase in bone mass in the treatment group. Other measurements of clinically significant increases in healing may include, for example, an assay for the N-terminal propeptide of Type I collagen, tests for breaking strength and tension, breaking strength and torsion, 4-point bending, increased connectivity in bone biopsies and other biomechanical tests well known to those skilled in the art. General guidance for treatment regimens is obtained from the experiments carried out in animal models of the disease of interest.

As used herein, “treatment” includes both prophylaxis and therapy. Thus, in treating a subject, the compounds of the invention may be administered to a subject already suffering from loss of bone mass or to prevent or inhibit the occurrence of such condition.

### DETAILED DESCRIPTION OF THE INVENTION

The present invention is based on applicants' discovery that certain forms of RANKL, which are exemplified by the GST-RANKL fusion product, cause an osteogenic response in osteoblasts. A RANK ligand that causes an osteogenic response is termed herein an osteogenic RANKL to distinguish it from naturally occurring RANKL which is an osteoclastic RANKL. Osteogenic RANKL can be identified by the response of osteoblasts. While qualitatively similar to that to native RANKL or an osteoclastic RANK ligand, the response of osteoblasts to an osteogenic RANKL differs in that its duration is longer. Osteoclastic RANKL causes an only transitory response in osteoblasts, while osteogenic RANKL causes a response that has a duration of at least thirty minutes.

Based on this discovery, the present invention provides for a method of discovery of other osteogenic forms of RANKL, which can be fusion proteins containing a fragment of the native RANKL protein or completely synthetic compounds that mimic the relevant structural features of RANKL. The method consists of the exposure of an osteoblast culture or osteoblast precursor culture to a candidate RANKL, and determining the duration and intensity of the response to the candidate and comparing the duration and intensity to that response to that caused by a known osteogenic RANKL.

The comparison step of the invention can be preferably performed directly, i.e., by comparing the culture's response to the candidate RANK ligand to that of GST-RANKL or other known osteogenic RANKL in a contemporaneous parallel culture. Alternatively, the comparison can be made with an historical control showing elevation that is comparable to that observed under the same conditions with the culture and a known osteogenic-RANKL.

In an alternative embodiment the comparison is performed longitudinally. Replicate cultures, i.e., at least duplicate, are established and the candidate compound is introduced into the cultures. The response of the cultures at time points that are shortly after the introduction and before and at or after one hour following the introduction is determined. An osteogenic RANKL can be identified by the persistence of the response by comparison to a contemporaneous control.

In accordance with the present invention, applicants have discovered that the interaction between RANKL and its receptor RANK on osteoblasts or osteoblast precursors results in accelerated rate of bone formation. Specifically, mice treated with a fusion product of RANKL (GST-RANKL) were shown to exhibit activation of osteoblasts and/or differentiation of osteoblast precursors.

Accordingly, the present invention provides methods of screening osteogenic compounds. Furthermore, the invention includes compounds that are identified by such screening methods. In addition, methods and compositions for enhancing bone formation are provided. These methods may be used to treat diseases or conditions, which are at least in part characterized by the loss of bone mass.

A. Screening by Bone Formation Assay

A method of screening osteogenic compounds comprises selecting a compound that activates some portion of the RANK pathway, performing a bone formation assay with said compound and determining the result of the bone formation assay, wherein a positive result indicates that the tested compound possesses osteogenic potential. The method may further include the step of identifying compounds that possess such potential. Alternatively, the sample material shown to possess osteogenic potential may be used directly without further isolation of its components.

1. Selecting RANK pathway activator

Briefly, selecting the compounds that bind to RANK, other proteins in the RANK pathway, or otherwise activate the RANK pathway, may be performed in multiple ways. The compounds may first be chosen based on their structural and functional characteristics, using one of a number of approaches known in the art. For instance, homology modeling can be used to screen small molecule libraries in order to determine which molecules would be candidates to interact with RANK, thereby selecting plausible targets. See http://www.neogenesis.com for a commercially available screening of compounds using multiple different approaches such as an automated ligand identification system and quantized surface complementarity. The compounds to be screened can include both natural and synthetic ligands. Furthermore, any desired compound may be examined for its ability to bind to RANK, as described below.

Binding to RANK or other proteins is determined by performing an assay such as, e.g., a binding assay between a desired compound and RANK. In one aspect, this is done by contacting said compound to RANK and determining its dissociation rate. Numerous possibilities for performing binding assays are well known in the art. The indication of a compound's ability to bind to RANK is determined, e.g., by a dissociation rate, and the correlation of binding activity and dissociation rates is well established in the art. For example, the assay may be performed by radio-labeling a reference compound, e.g. RANKL with 125I and incubating it with RANK in 1.5 ml tubes. Test compounds are then added to these reactions in increasing concentrations. After optimal incubation, the RANK/compound complexes are separated, e.g., with chromatography columns, and evaluated for bound ¹²⁵I-labeled peptide with gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek et al., Proc. Natl. Acad. Sci. USA, Vol. 97, No. 21, pp. 11460-11465, 2000. In preferred embodiments, the radiolabeled RANK ligand whose binding is measured in the presence of screened compounds may comprise a surface contact loop of RANKL, or a fragment, derivative, or analog thereof, specifically including, without limitation, those polypeptides identified as SEQ ID NO. 1 through 5 of U.S. provisional application Serial No. 60/329,360 filed Oct. 15, 2001 and U.S. non-provisional entitled Bone Anti-Resorptive Compounds filed Oct. 12, 2002.

The compounds may also be selected from libraries of compounds or by designing suitable RANKL mimics. Various high throughput assays, as discussed elsewhere in this application, are known and may be used to perform the assays involving the test mimics.

A RANKL mimic may be computationally evaluated and designed by means of a series of steps in which chemical groups or fragments are screened and selected for their ability to associate with the individual binding pockets or interface surfaces of each of the proteins. One skilled in the art may use one of several methods to screen chemical groups or fragments for their ability to associate with RANK. This process may begin by visual inspection of, for example, the protein/protein interfaces or the binding site on the computer screen based on the crystal complex coordinates. Selected fragments or chemical groups may then be positioned in a variety of orientations, or docked, at an individual surface of RANKL that participates in a protein/protein interface or in the binding pocket. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical groups. These include:

1. GRID (Goodford, 1985, J. Med. Chem. 28:849-857). GRID is available from Oxford University, Oxford, UK;

2. MCSS (Miranker & Karplus, 1991, Proteins: Structure, Function and Genetics 11:29-34). MCSS is available from Molecular Simulations, Burlington, Mass.;

3. AUTODOCK (Goodsell & Olsen, 1990, Proteins: Structure, Function, and Genetics 8:195-202). AUTODOCK is available from Scripps Research Institute, La Jolla, Calif.; and

4. DOCK (Kuntz et al., 1982, J. Mol. Biol. 161:269-288). DOCK is available from University of California, San Francisco, Calif.

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates thereof. This would be followed by manual model building using software such as QUANTA or SYBYL.

Useful programs to aid one of skill in the art in connecting the individual chemical groups or fragments include:

1. CAVEAT (Bartlett et al., 1989, ‘CAVEAT: A Program to Facilitate the Structure-Derived Design of Biologically Active Molecules’. In Molecular Recognition in Chemical and Biological Problems’, Special Pub., Royal Chem. Soc. 78:182-196). CAVEAT is available from the University of California, Berkeley, CA;

2. 3D Database systems such as MACCS-3D (MDL Information Systems, San Leandro, Calif.). This area is reviewed in Martin, 1992, J. Med. Chem. 35:2145-2154); and

3. HOOK (available from Molecular Simulations, Burlington, Mass.).

Instead of proceeding to build a RANKL mimic, in a step-wise fashion one fragment or chemical group at a time, as described above, such compounds may be designed as a whole or ‘de novo’ using either an empty binding site or the surface of a protein that participates in protein/protein interactions or optionally including some portion(s) of a known activator(s). These methods include:

1. LUDI (Bohm, 1992, J. Comp. Aid. Molec. Design 6:61-78). LUDI is available from Molecular Simulations, Inc., San Diego, Calif.;

2. LEGEND (Nishibata & Itai, 1991, Tetrahedron 47:8985). LEGEND is available from Molecular Simulations, Burlington, Mass.; and

3. LeapFrog (available from Tripos, Inc., St. Louis, Mo.).

Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210.

Once a compound has been designed by the above methods, the efficiency with which that compound may bind to RANK or other proteins may be tested and optimized by computational evaluation. Inhibitors may interact with the protein in more than one conformation that is similar in overall binding energy. In those cases, the deformation energy of binding is taken to be the difference between the energy of the free compound and the average energy of the conformations observed when the inhibitor binds to the protein.

A compound selected or designed for binding to RANK or other proteins may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the protein when the mimic is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

Once a RANKL mimic compound has been optimally designed, as described above, substitutions may then be made in some of its atoms or chemical groups in order to improve or modify its binding properties, or its pharmaceutical properties such as stability or toxicity. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to RANK by the same computer methods described in detail above.

Because RANKL/RANK may crystallize in more than one crystal form, the structure coordinates of RANKL complexes, or of portions thereof, are particularly useful to solve the structure of those other crystal forms. They may also be used to solve the crystal structure of any other protein or protein complex with significant amino acid sequence homology to any functional domain of RANKL. The RANKL coordinates are available for the Protein Data Bank under the accession code 1JTZ.

In addition, phage display libraries may be used to screen potential ligands. Their usefulness lies in the ability to screen, for example, a library displaying a billion different compounds with only a modest investment of time, money, and resources. For use of phage display libraries in a screening process, see, for instance, Kay et al., Methods, 240-246, 2001. An exemplary scheme for using phage display libraries may be described as follows: initially, an aliquot of the library is introduced into microtiter plate wells that have previously been coated with target protein, e.g. RANK. After a 2-h incubation, the nonbinding phage are washed away, and the bound phage are recovered by denaturing or destroying the target with exposure to harsh conditions such as, for instance pH 2, but leaving the phage intact. After transferring the phage to another tube, the conditions are neutralized, followed by infection of bacteria with the phage and production of more phage particles. The amplified phage are then rescreened to complete one cycle of affinity selection. After three or more rounds of screening, the phage are plated out such that there are individual plaques that can be further analyzed. For example, the conformation of binding activity of affinity-purified phage for RANK may be obtained by performing ELISAs. One skilled in the art can easily perform these experiments.

In one aspect, a RANK molecule used for any of the assays may be selected from a recombinant RANK protein, a RANK fusion protein, an analog, derivative, or mimic thereof. In a preferred aspect, RANK is a recombinant RANK protein.

2. Performing Bone Formation Assay and Determining Results

Once a compound that binds to RANK or otherwise activates the RANK pathway is selected, it is then tested for its osteogenic potential. There are multiple bone formation assays that can be used successfully to screen potential osteogenic compounds of this invention. See, e.g., U.S. Pat. No. 6,080,779.

One commonly used assay is a neonatal mouse calvaria assay. Briefly, four days after birth, the front and parietal bones of ICR Swiss white mouse pups are removed by microdissection and split along the sagittal suture. The bones are then incubated in a specified medium, wherein the medium contains either test or control compounds. Following the incubation, the bones are removed from the media, and fixed in 10% buffered formalin for 24-48 hours, decalcified in 14% EDTA for 1 week, processed through graded alcohols, and embedded in paraffin wax. Three micron sections of the calvaria are prepared and assessed using histomorphometric analysis of bone formation or bone resorption. Bone changes are measured on sections cut 200 microns apart. Osteoblasts and osteoclasts are identified by their distinctive morphology.

In addition to this assay, the effect of compounds on murine calvarial bone growth can also be tested in vivo. In one such example of this screening assay, male ICR Swiss white mice, aged 4-6 weeks are employed, using 4-5 mice per group. Briefly, the test compound or the appropriate control is injected into subcutaneous tissue over the right calvaria of normal mice. The mice are sacrificed on day 14, and bone growth is measured by histomorphometric means. Bone samples are cleaned from adjacent tissues and fixed in 10% buffered formalin for 24-48 hours, decalcified in 14% EDTA for 1-3 weeks, processed through graded alcohols, and embedded in paraffin wax. Three to five micron sections of the calvaria are prepared, and representative sections are selected for histomorphometric assessment of the effects of bone formation and bone resorption. Sections are measured by using a camera lucida attachment to trace directly the microscopic image onto a digitizing plate. Bone changes are measured on sections cut 200 microns apart, over 4 adjacent 1*1 mm fields on both the injected and noninjected sides of calvaria. New bone may be identified by those skilled in the art by its characteristic tinctorial features, and osteoclasts and osteoblasts may be identified by their distinctive morphology. Histomorphometry software (OsteoMeasure, Osteometrix, Inc., Atlanta) can be used to process digitized input to determine cell counts and measure areas or perimeters.

Additional in vivo assays include dosing assays in intact animals, and dosing assays in acute ovariectomized (OVX) animals (prevention model), and assays in chronic OVX animals (treatment model). Prototypical dosing in intact animals may be accomplished by subcutaneous, intraperitoneal or oral administration, and may be performed by injection, sustained release or other delivery techniques. The time period for administration of test compound may vary (for instance, 28 days as well as 35 days may be appropriate). As an example, in vivo oral or subcutaneous dosing assay may be performed as described below.

In a typical study, 70 three-month-old female Sprague-Dawley rats are weight-matched and divided into seven groups, with ten animals in each group. This includes a baseline control group of animals sacrificed at the initiation of the study; a control group administered vehicle only; a PBS-treated control group; and a positive group administered a compound known to enhance bone mass. Three dosage levels of the test compound are administered to the remaining groups. Test compound, PBS, and vehicle are administered subcutaneously once per day for 35 days. All animals are injected calcein nine days and two days before sacrifice (to ensure proper labeling of newly formed bone). Weekly body weights are determined. At the end of 35 days, the animals are weighed and bled by orbital or cardiac puncture. Serum calcium, phosphate, osteocalcin, and CBCs are determined. Both leg bones (femur and tibia) and lumbar vertabrae are removed, cleaned of adhering soft tissue, and stored in 70% ethanol or 10% formalin for evaluation, as performed by peripheral quantitative computed tomography (pQCT; Ferretti, J, Bone, 17: 353S-364S, 1995), dual energy X-ray absorptiometry (DEXA; Laval-Jeantet A. et al., Calcif Tissue Intl, 56:14-18, 1995, and Casez J. et al., Bone and Mineral, 26:61-68, 1994) and/or histomorphometry. The effect of test compounds on bone remodeling can thus be evaluated.

Test compounds can also be assayed in acute ovariectomized animals. Such assays may also include an estrogen-treated group as a control. An example of the test in these animals is briefly described below.

In a typical study, 80 three-month-old female Sprague-Dawley rats are weight-matched and divided into eight groups, with ten animals in each group. This includes a baseline control group of animals sacrificed at the initiation of the study; three control groups (sham OVX and vehicle only, OVX and vehicle only, and OVX and PBS only); and a control OVX group that is administered a compound known to enhance bone mass. Three dosage levels of the test compound are administered to remaining groups of OVX animals.

Since ovariectomy induces hyperphagia, all OVX animals are pair-fed with sham OVX animals throughout the 35 day study. Test compound, positive control compound, PBS or vehicle alone is administered orally or subcutaneously once per day for 35 days. As an alternative, test compounds can be formulated in implantable pellets that are implanted for 35 days, or may be administered orally, such as by gastric gavage. All animals are injected with calcein nine days and two days before sacrifice. Weekly body weights are determined. At the end of the 35-day cycle, the animals blood and tissues are processed as described above.

Test compounds may also be assayed in chronic OVX animals. Briefly, 80 to 100 six month old female, Sprague-Dawley rats are subjected to sham surgery (sham OVX), or ovariectomy (OVX) at the beginning of the experiment, and 10 animals are sacrificed at the same time to serve as baseline controls. Body weights are monitored weekly. After approximately six weeks or more of bone depletion, 10 sham OVX and 10 OVX rats are randomly selected for sacrifice as depletion period controls. Of the remaining animals, 10 sham OVX and 10 OVX rats are used as placebo-treated controls. The remaining animals are treated with 3 to 5 doses of test compound for a period of 35 days. As a positive control, a group of OVX rats can be treated with a known anabolic agent in this model, such as PTH (Kimmel et al., Endocrinology, 132: 1577-1584, 1993). At the end of the experiment, the animals are sacrificed and femurs, tibiae, and lumbar vertebrael to 4 are excised and collected. The proximal left and right tibiae are used for pQCT measurements, cancellous bone mineral density (BMD), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD (pQCT may also be performed), LV3 are prepared for undecalcified bone histology, and LV4 are processed for mechanical testing.

In a preferred embodiment, bone forming ability of a compound is tested in an in vitro assay utilizing osteoblasts or osteoblast-like cells. A general protocol for treatment of osteoblasts with a compound is well established in the art. See, for instance, Wyatt et al., BMC Cell Biology, 2:14, 2001. A cell line of choice in this article was MC3T3-E1, which has been used as an in vitro model of osteoblastic differentiation and maturation. The treatment of cells, in this case with BMP2, was performed in the following manner. The cells were plated at 5000/cm² in plastic 25 cm² culture flasks in α-MEM supplemented with 5% fetal bovine serum, 26 mM NaHCO₃, 2 mM glutamine, 100 u/ml penicillin, and 100 μg/ml streptomycin, and grown in humidified 5% CO₂/95% air at 37° C. Cells were passaged every 3-4 days after releasing with 0.002% pronase E in PBS. The cells in treatment groups were grown for 24 hours, then incubated with BMP-2 (50 ng/ml) dissolved in PBS containing 4 mM HCI and 0.1% bovine serum albumin (BSA) at 37° C. for 24 and 48 hours. Control groups received equal volumes of vehicles only. Exemplary conditions for treatment of osteoblast cells or precursors with a compound that binds to RANK such as, e.g., GST-RANKL are described below. Osteoblast precursor cells are incubated in the presence of vehicle, GST (a negative control), or increasing concentrations of purified GST-RANKL (e.g. concentrations ranging from 1 ng/ml to 100 ng/ml). Bone morphogenetic protein (BMP)-2 was administered as a positive control. Test compositions were administered for a period of 12 hours only at the initiation of the culture or once at initiation and once three days later, again for a duration of 12 hours. It is to be noted that the conditions used will vary according to the cell lines and compound used, their respective amounts, and additional factors such as plating conditions and media composition. Such adjustments are readily determined by one skilled in this art.

The activation of osteoblasts or differentiation of osteoblast precursors may be determined by assessing the activation of intracellular proteins indicative of bone formation, particularly kinases such as ERK, JNK, p38, Akt, PI3K and IKK. See, U.S. Application Serial No. 60/328,876 filed Oct. 12, 2001 and U.S. non-provisional application entitled RANKL Mimics and Uses Thereof filed Oct. 12, 2002. Thus, following the incubation of osteoblasts with a test compound, the cells are lysed and their intracellular contents subjected to the appropriate tests, such as Western blots, kinase assays, and electrophoretic mobility gel shift assays (EMSAs).

A Western blot can be generally performed as follows. Once the cell lysates are generated, the intracellular proteins are separated on the basis of size by utilizing SDS-PAGE (sodium dodecyl sulfate-polyacrylamide gel electrophoresis). The separated proteins are transferred by electroblotting to a suitable membrane (such as nitrocellulose or polyvinylidene flouride) to which they adhere. The membrane is washed to reduce non-specific signals, and then probed with an antibody which recognizes only the specific amino acid which has been phosphorylated as a result of RANK signaling. After further washing, which removes excess antibody, a second antibody, which recognizes the first antibody (bound to specifically-phosphorylated proteins on the membrane) and contains a reporter moiety is applied to the membrane. The addition of a developing agent, which interacts with a reporter moiety on the second antibody results in visualization of the bands.

A kinase assay, for example for ERK1/2, can be performed by utilizing a known substrate for this kinase such as p90 ribosomal S6 protein kinase (RSK). Briefly, by way of example, treated osteoblasts are washed in ice-cold PBS, e.g., three times, and extracted with lysis buffer in order to obtain cell lysates. Supernatants obtained after microcentifugation of cell lysates are incubated with goat anti-RSK2 antibody (1:200) together with protein G-Sepharose at 4° C. overnight. The beads are collected by microcentrifugation, washed twice with lysis buffer, followed by kinase buffer. RSK2 phosphotransferase activity in the beads is measured by using S6 kinase assay kit and [γ-³²P]ATP according to the protocols provided by the manufacturer (Upstate Biotechnology, Inc).

An additional assay that can be applied to determine activation of osteoblasts is an electrophoretic mobility gel shift assay (EMSA). This assay monitors nuclear translocation of a transcription factor complex (such as NFKB following activation of osteoblasts with GST-RANKL). Briefly, an EMSA may be conducted as follows. Nuclei of treated osteoblasts are isolated and their extracts generated. The nuclear proteins are then incubated with a specific oligonucleotide probe that has been labeled with ³²p orthophosphate. After an appropriate time, the putative protein-DNA complexes are separated on a PAGE gel (no SDS present), which is dried and exposed to an X-ray film. If a specific complex has formed (in this case a complex of NFKB proteins with a specific DNA sequence) a band will be visible on the developed film. Typically, appropriate controls are run in parallel with the experimental sample(s) in order to ensure that the band is specific for activated osteoblasts. For detailed procedures on Western blotting, kinase assays, and EMSA, see for example Lai et al., (Journal of Biological Chemistry), 276(17):14443-14450, Apr. 27, 2001. Additionally, cell-based assays for osteoblast differentiation and function, based on measuring collagen levels and alkaline phosphatase activity may be used. These assays are well known in the art and easily performed by a skilled artisan.

B. Screening by Determining Activation of Osteoblasts and/or Osteoblast Intracellular Pathways

In accordance with the present invention, applicants have discovered that the interaction between GST-RANKL and its receptor RANK on osteoblasts or osteoblast precursors results in accelerated rate of bone formation. Using in vitro assays, mouse osteoblasts, when treated with GST-RANKL, manifested activation, as characterized by the activation of the RANK pathway, specifically, the NF_{K}B and MAP Kinase intracellular signal pathways, including ERK1/2, p38, and JNK. As noted by the applicants, the time course of intracellular protein activation, especially ERK activation is different from that observed in osteoclasts, which also express RANK on the surface. In osteoclasts, ERK activation peaks 5-15 minutes after RANK/RANKL interaction, and returns to basal levels within 15-30 minutes.

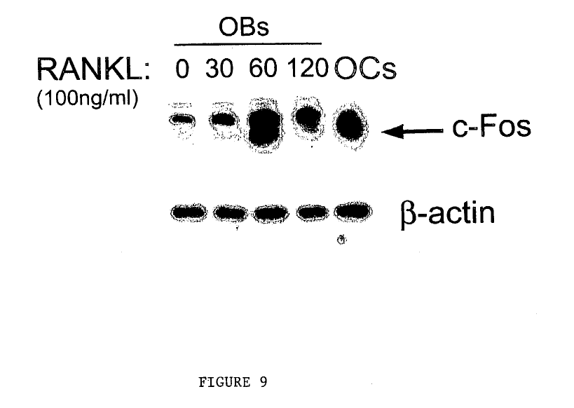

In contrast, the ERK activation in osteoblasts peaks at 10 minutes after the same interaction, and is still above the basal level for up to 60 minutes. The prolongation of the time course is even more prominent in osteoblast precursor cells, wherein the demonstrated activation of ERK had not reached its maximum even 60 minutes after the RANK/GST-RANKL interaction. Besides the different time course of ERK activation, osteoblasts and osteoblast precursor cells also exhibit prolonged activation of c-Fos (See FIG. 9) as well as of kinases such as IKK, PI3 kinase, Akt, p38 and JNK. This osteoblast-related activity contrasts with RANK ligand interaction with RANK on osteoclasts, which results in short-lived activation of MAP kinases and in bone resorption. While not being bound to a particular theory, it therefore appears that the prolonged activation of kinases observed in osteoblasts following GST-RANKL stimulation plays a role in the process opposite to bone resorption, i.e. bone formation.

Accordingly, the present invention provides methods for screening osteogenic compounds by determining activation of osteoblasts and/or osteoblast intracellular pathways related to bone formation, including the RANK pathways. The invention also includes compounds that are identified by the screening methods disclosed herein. Further provided are methods and compositions for stimulating bone formation.

Accordingly, the methods of the present invention allow for identification of compounds that can be used to treat diseases or conditions characterized at least in part by the loss of bone mass. It is to be noted that the methods for screening osteogenic compounds may be performed with either osteoblasts or osteoblast precursors since both cell types exhibit prolonged activation of the same kinases following stimulation with GST-RANKL. Therefore, methods described herein that utilize osteoblasts can also be performed with osteoblast precursors.

In one embodiment, the method of screening osteogenic compounds involves incubating a test compound with osteoblasts or osteoblast-like cells under conditions sufficient for such incubation. The test compound may be a compound that binds to RANK. Selecting the compounds that bind to RANK may be performed in multiple ways as described above and herein. The compound may also not bind to RANK, but may stimulate one or more of the proteins in the RANK pathway downstream of RANK.

Another embodiment of the present invention involves use of the RANKL loops in competitive binding assays to screen for inhibitors of RANKL. Binding to RANK is determined by performing an assay as described above as a binding assay between a desired compound and RANK. In one aspect, this is done by contacting a test compound to RANK and determining its dissociation rate. Numerous possibilities for performing binding assays are well known in the art. The indication of a compound's ability to bind to RANK is determined, e.g., by a dissociation rate, and the correlation of binding activity and dissociation rates is well established in the art. For example, the assay may be performed by radio-labeling a reference compound, e.g. a polypeptide containing a portion of AA” loop sequence SEQ ID NO 1 of U.S. Application Serial No. 60/329,360 and U.S. non-provisional Application entitled Bone Anti-Resorptive Compounds filed Oct. 12, 2002 with ¹²⁵I and incubating it with RANK in 1.5 ml tubes. Test compounds are then added to these reactions in increasing concentrations. After optimal incubation, the RANK/compound complexes are separated, e.g., with chromatography columns, and evaluated for bound ¹²⁵I-labeled peptide with gamma (γ) counter. The amount of the test compound necessary to inhibit 50% of the reference compound's binding is determined. These values are then normalized to the concentration of unlabeled reference compound's binding (relative inhibitory concentration (RIC)-¹=concentrationₜₑₛₜ/concentration_{reference}). A small RIC⁻¹ value indicates strong relative binding, whereas a large RIC-¹ value indicates weak relative binding. See, for example, Latek et al., Proc. Natl. Acad. Sci. USA, Vol. 97, No. 21, pp. 11460-11465, 2000. The RANKL loops identified in SEQ ID NO 2-5 of U.S. Application Serial No. 60/329,360 and U.S. non-provisional Application entitled Bone Anti-Resorptive Compounds filed Oct. 12, 2002 are also suitable for use as a reference compound in such assays. Again, high throughput assays are also suitable to perform the binding assays involving the RANKL loops.

General protocols and assays for the treatment of osteoblasts with a compound are also described above. Similarly, activation of osteoblasts and/or their intracellular compounds related to bone formation, may be performed as described above. For purposes of the present embodiment, the assays may consist of determining the activation of intracellular proteins correlated with bone formation. These proteins include but are not limited to kinases. Preferably, the kinases include ERK1/2, JNK, PI3 kinase, Akt, and p38. More preferably, the readout of the assay is determined by measuring the activation of ERK1/2. Other molecules that may be assayed belong to NF_{K}B signaling pathway due to the fact that NF_{K}B is involved in osteoblast activation and osteoblast precursor differentiation. One such molecule is a kinase, IKK. NF_{K}B is normally sequestered in cytoplasm of resting cells by IkB which binds to it and masks its nuclear localization signal. Upon exposure of cells to certain stimuli, such as GST-RANKL stimulation of osteoblasts, IKK phosphorylates IkB, which frees NF_{K}B so that it can translocate into the nucleus and regulate gene transcription. Phosphorylated IkB serves as a target for ubiquitin ligase, thereby leading to ubiquitination and degradation of NF_{K}B. As can be seen from this pathway, numerous molecules are involved in NF_{K}B regulation. Thus, in addition to IKK, all of these molecules represent credible targets for activation assays.

In another preferred embodiment, the activation comprises phosphorylation of intracellular proteins in the RANK pathway, and more preferably of kinases. For the MAP kinase family, full activation requires dual phosphorylation on tyrosine and threonine residues separated by a glutamate residue (known as TEY motif) by a single upstream kinase known as MAP kinase kinase (MKK). The requirement for dual phosphorylation ensures that MAP kinases are specifically activated by the action of MKK. The methods of the present invention may utilize any of the appropriate assays available in the art for determining whether a kinase has been phosphorylated. Preferably, the assays used are Western blots or kinase assays.

Applicants have discovered that osteoblasts activated through GST-RANKL stimulation exhibit prolonged kinase activation. The activation in osteoblasts can be detected up to at least 60 minutes of RANK/GST-RANKL interaction. In osteoblast precursor cells, the activation peaks after 5-10 minutes, and can be detected for as long as 60 minutes. Accordingly, the activation of intracellular proteins in screening osteogenic compounds may be detected for at least about 30 minutes after the incubation of the test compound with osteoblasts or osteoblast precursors. In a preferred embodiment, the activation is detected for at least about 40 minutes, and more preferably for at least about 60 minutes after said incubation. In another preferred embodiment, the intracellular proteins whose activation is detected for at least about 30 minutes are kinases, and more preferably, the kinase is ERK1/2. Activation of additional kinases in this pathway may also be assayed, for example those involved in phosphorylating NFKB within the nucleus.

The compound that activates osteoblasts and/or stimulates differentiation of osteoblast precursors can then be tested in a bone formation assay as described above, wherein an increase in bone mass over the increase in background bone mass designates a compound as having osteogenic activity.

C. Screening by Determining Phosphatase Inactivation Activity

In another embodiment, methods for screening osteogenic compounds based on their ability to inactivate phosphatase(s) (partially or completely) are also provided herein. The compounds to be screened may include compounds that bind to RANK, and methods for selecting such compounds are described above. The phosphatases inhibit the kinases specific for osteogenesis, including p38, ERKs, JNK, IKK, and Akt. Preferably, the phosphatases are MAPK specific or Akt specific, and even more preferably they are ERK1/2 specific. While not being bound to a particular theory, this method is feasible for this purpose due to the fact that a kinase activity is tightly regulated by its corresponding phosphatase. In case of ERK1/2, the phosphatase is known as the mitogen activated protein kinase phosphatase-3 (MKP-3). This phosphatase belongs to a family of dual specificity phosphatases, which are responsible for the removal of phosphate groups from the threonine and tyrosine residues on their corresponding kinases (Camps et al., FASEB J., 14, pp. 6-16, 1999). The prompt removal of phosphate groups by phosphatases ensures that kinase activation is short-lived and that the level of phosphorylation is low in a resting cell. However, in order for the phosphatase to be active and remove phosphate groups, it also needs to be phosphorylated. Therefore, inhibition (dephosphorylation) of phosphatase activity results in activation or prolongation of ERK1/2 activity.

This method of screening involves initially activating osteoblasts with a substance known to activate these cells, such as GST-RANKL or BMP-2 (bone morphogenetic protein 2). This leads to activation of phosphatases, at which point osteoblasts are treated with a test compound and cell lysates are obtained. The ability of the test compound to dephosphorylate (inactivate) phosphatase(s) is determined by performing Western blots or kinase assays. See above. For additional details on assessing phosphatase activity, see Muda et al., J Biol Chem., 273:9323-9329, 1998, and Camps et al., Science 280:1262-1265, 1998. If the compound is determined to possess phosphatase inhibitory activity, it can further be tested in one of the bone formation assays to determine its osteogenic activity. These assays were described above.

Additional phosphatases in this pathway may also be assayed, for example those involved in dephosphorylating NFKB within the nucleus.

D. Screening by Nuclear Translocation of NFKB Family Transcription Factors In another embodiment, methods for screening osteogenic compounds include measuring the nuclear translocation of NFKB family proteins, utilizing EMSA, as in Mayo et al., JBC 277: 1116-1126, 2002 and references therein, or other methods well known in the art.

E. Identification of Screened Osteogenic Compounds

The methods of screening osteogenic compounds may also include the identification of osteogenic compounds, whose osteogenic potential was determined by the methods described above. If the identity of the compound is known from the start of the experiment, no additional assays are needed to determine its identity. However, if the screening for compounds that bind to RANK is done with a library of compounds, it may be necessary to perform additional tests to positively identify a compound that satisfies all required conditions of the screening process. There are multiple ways to determine the identity of the compound. One process involves mass spectrometry, available from Neogenesis. See http://www.neogenesis.com. Neogenesis' ALIS (automated ligand identification system) spectral search engine and data analysis software allow for a highly specific identification of a ligand structure based on the exact mass of the ligand. One skilled in the art can also readily perform mass spectrometry experiments to determine the identity of the compound.

F. Method of Using Screened Compounds

In a preferred embodiment of the invention, a method of preventing or inhibiting bone loss or of enhancing bone formation is provided by administering compositions comprising compounds identified by the screening methods provided herein. The bone forming compositions of the present invention may be utilized by providing an effective amount of such compositions to a subject in need thereof.

For use in treatment of animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The administration of the compositions of the present invention may be pharmacokinetically and pharmacodynamically controlled by calibrating various parameters of administration, including the frequency, dosage, duration mode and route of administration. Thus, in one embodiment bone mass formation is achieved by administering a bone forming composition in a non-continuous, intermittent manner, such as by daily injection and/or ingestion. Variations in the dosage, duration and mode of administration may also be manipulated to produce the activity required.

For administration to animal or human subjects, the dosage of the compounds of the invention is typically 0.01100 mg/kg. However, dosage levels are highly dependent on the nature of the disease or situation, the condition of the subject, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary.

It will be understood that the appropriate dosage of the substance should suitably be assessed by performing animal model tests, wherein the effective dose level (e.g. ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

In general, for use in treatment, the compounds of the invention may be used alone or in combination with other compositions for the treatment of bone loss. Such compositions include anti-resorptives such as a bisphosphonate, a calcitonin, a calcitriol, an estrogen, selective estrogen receptor modulators (SERM's) and a calcium source, or a supplemental bone formation agent like parathyroid hormone or its derivative, a bone morphogenetic protein, osteogenin, NaF, or a statin. See U.S. Pat. No. 6,080,779 incorporated herein by reference. Depending on the mode of administration, the compounds will be formulated into suitable compositions.

Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, nasal, or oral administration. The formulation will generally include a diluent as well as, in some cases, adjuvants, buffers, preservatives and the like.

For oral administration, the compositions can be administered also in liposomal compositions or as microemulsions. Suitable forms include syrups, capsules, tablets, as is understood in the art. For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

The compositions of the present invention may also be administered locally to sites in subjects, both human and other vertebrates, such as domestic animals, rodents and livestock, where bone formation and growth are desired using a variety of techniques known to those skilled in the art. For example, these may include sprays, lotions, gels or other vehicles such as alcohols, polyglycols, esters, oils and silicones. Such local applications include, for example, at a site of a bone fracture or defect to repair or replace damaged bone. Additionally, a bone forming composition may be administered e.g., in a suitable carrier, at a junction of an autograft, allograft or prosthesis and native bone to assist in binding of the graft or prosthesis to the native bone.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

### Example 1

Ex vivo stimulation of bone formation in whole calvarial organ culture. An assay for bone formation was carried out as described in U.S. Pat. No. 6,080,779 col. 10, II. 29-55 incorporated herein by reference. Neo-natal mouse calvariae were placed in organ culture in the presence of vehicle, GST (a negative control), bone morphogenetic protein (BMP)-2 (a positive control) or increasing concentrations of purified GST-RANKL obtained as outlined briefly below. CDNA encoding murine RANKL residues 158-316 was cloned into pGEX-4T-1 (Amersham, GenBank Accession No. U13853) downstream of glutathione S-transferase (GST), sequence. Following IPTG-mediated induction of protein expression in BL21 (DE3) Escherichia coli (Invitrogen), cells were lysed and GST-RANKL fusion protein was affinity purified from the cell lysates incubated with glutathione sepharose (Amersham). GST-RANKL was eluted for the affinity column, subjected to ion exchange chromatography, and dialyzed against physiologic salt and pH. Purified GST-RANKL was then assayed for endotoxin contamination by limulus amoebocyte lysate assay, and quantitated for bioactivity by an in vitro osteoclastogenesis readout.

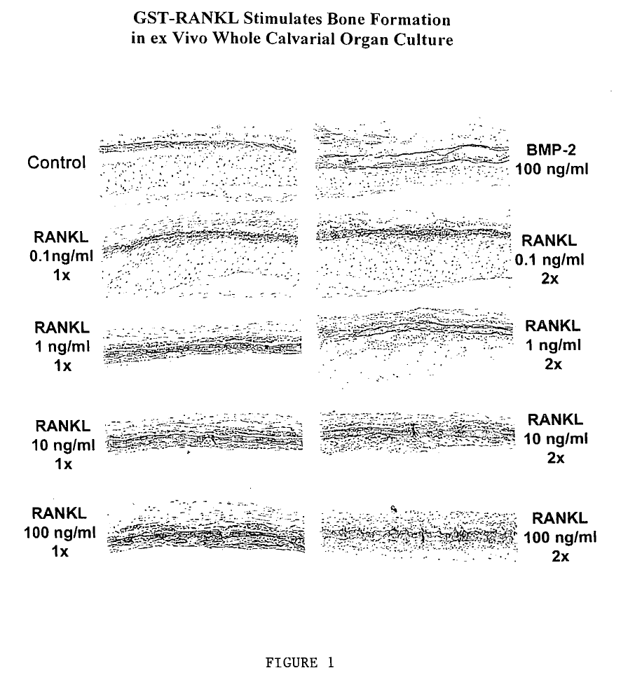

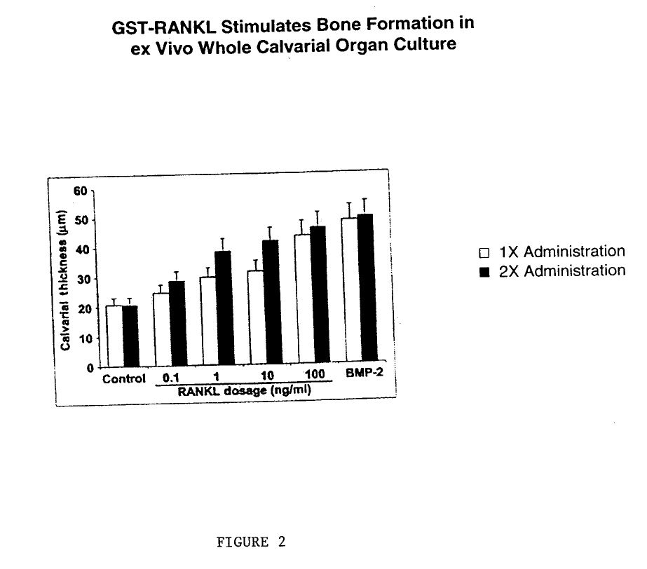

Test compositions were administered for-a period of 12 hours only at the initiation of the culture (1×) or once at initiation and once three days later, again for a duration of 12 hours (2×). After seven days, calvarial thickness was determined histomorphometrically and compared among the various control and experimental groups to assess bone formation. See FIG. 1. GST-RANKL induced a dose-dependent increase in cavarial thickness when administered 1× or 2×. See FIG. 2. At the highest doses tested (100 ng/ml) calvarial thickness had doubled.

### Example 2

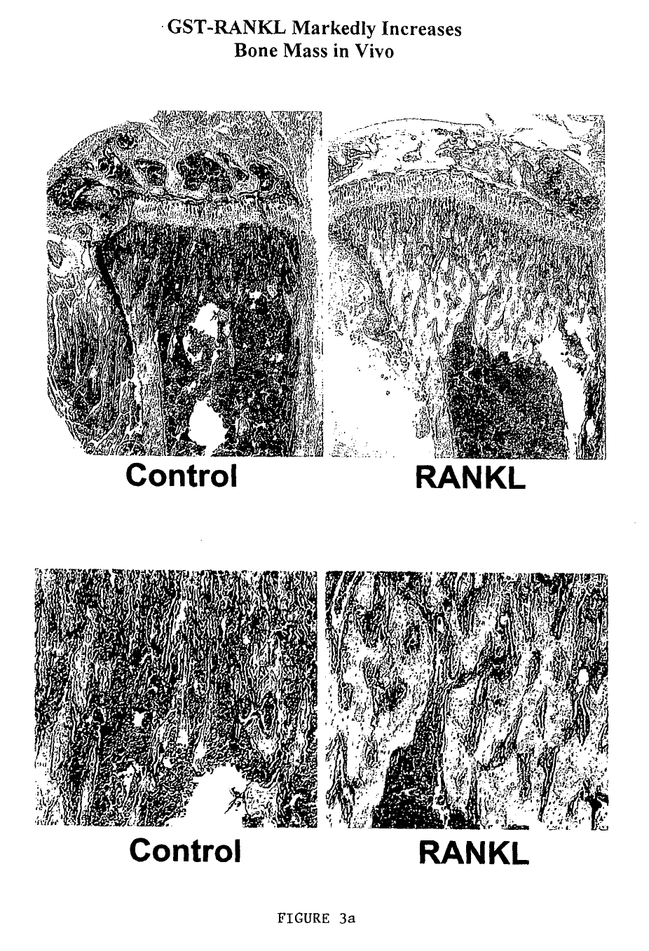

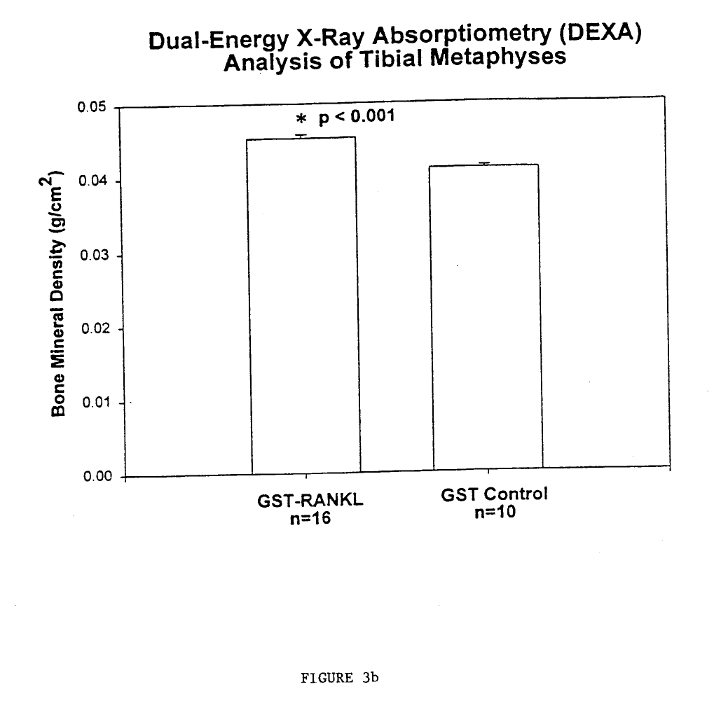

In vivo stimulation of bone formation in mice. Mice, C3H/HeN (Harlan, Indianapolis, Ind.) were administered 100 micrograms GST (control) or 100 micrograms GST-RANKL as obtained in Example 1, subcutaneously, once a day for nine days. Histological examination of tibia reveals a marked increase in bone mass and a net increase in the numbers of activated osteoblasts in GST-RANKL-treated as compared to control mice. See FIG. 3(a). Upper panels illustrate metaphyseal bone, which is shown at higher magnification in lower panels. The upper, low magnification panels show internal and external surfaces of cortex, documenting massive increase in cortical thickness in GST-RANKL treated mice, as well as active osteoblasts lining newly-formed bone. Dual-energy X-ray absorptiometry (DEXA) analysis of GST or GST-RANKL administered mice was also conducted using standard procedures. Results (see FIG. 3(b)) show a significant increase in bone mineral density of GST-RANKL compared to control.

### Example 3

AP activity following RANKL exposure in osteoblasts. Primary calvarial osteoblasts were cultured in MEM containing 15% FBS, 50 μM ascorbic acid, and 10 mM β-glycerophosphate. Cells were maintained at 37° C., with daily replenishment of media and cytokines. Osteoblast alkaline phosphatase (AP) activity, a direct measure of osteoblast differentiation and function, was quantitated by addition of a calorimetric substrate, 5.5 mM p-nitrophenyl phosphate. The cells were then exposed to GST-RANKL, administered in different regimens. Pulsatile exposure to 50 ng/ml GST-RANKL was provided as 1, 3, 6, 8, or 24 hours of total exposure per 48-hour treatment window. After 4 such 48-hour treatments, AP activity was quantitated (±S.D.) and normalized to total protein levels.

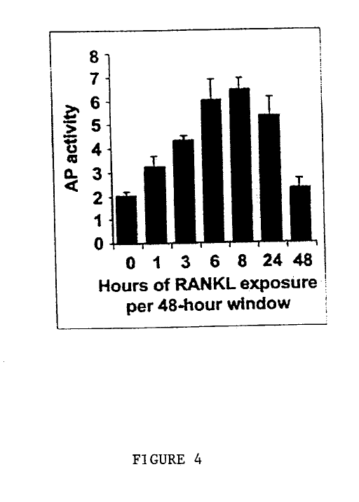

As can be seen from FIG. 4, the maximum anabolic effect was observed when RANKL exposure was provided for an 8-hour treatment window, once every 48 hours. Thus, GST-RANKL induced increase in AP activity when administered in an intermittent fashion.

### Example 4

GST-RANKL rapidly activates MAP kinases in murine osteoclast precursors. Wild type C57BL/6 mice were purchased from Harlan Industries (Indianapolis, Ind.). For the isolation of osteoclast precursors, bone marrow macrophages (BMMs) were isolated from whole bone marrow of four to six week old mice and incubated in tissue culture dishes at 37° C. in 5% CO₂. After 24 hours in culture, the non-adherent cells were collected and layered on a Ficoll Hypaque gradient and the cells at the gradient interface were collected. Cells were replated at 65,000/cm² in α-minimal essential medium, supplemented with 10% heat inactivated fetal bovine serum, at 37° C. in 5% CO₂ in the presence of recombinant mouse M-CSF (10 ng.ml). Cells were treated with GST-RANKL on day 4 or 5. In the experiments addressing the activation of Akt, the cells were cultured in serum and M-CSF free medium for 24 hours prior to GST-RANKL stimulation.

Immunoblotting (Western blotting) of osteoclast precursors was performed according to the following instructions. Cytokine-treated or control monolayers of BMMs were washed twice with ice-cold PBS. Cells were lysed in the buffer containing 20 mM Tris (pH 7.5),150 mM NaCl, 1 mM EDTA, 1 mM EGTA, 1% Triton X-100, 2.5 mM sodium pyrophoshate, 1 mM β-glycerophosphate, 1 mM Na₃PO₄, 1 mM NaF, and 1×protease inhibitor cocktail. Fifty μg of cell lysates were boiled in the presence of SDS sample buffer (0.5 M Tris-HCl, pH 6.8, 10% w/v SDS, 10% glycerol, 0.05% w/v bromphenol blue) for 5 minutes and separated on SDS-PAGE, using 8% gels. Proteins were transferred to nitrocellulose membranes using a semi-dry blotter (Bio-Rad, Richmond, Calif.) and incubated in blocking solution (5% non-fat dry milk in tris-buffered saline containing 0.1% Tween 20) for 1 hour to reduce nonspecific binding. Membranes were then exposed to primary antibodies overnight at 4° C., washed three times, and incubated with secondary goat anti-mouse or rabbit IgG horseradish peroxidase-conjugated antibody for 1 hour. Membranes were washed extensively, and enhanced chemiluminescence detection assay was performed following the manufacturer's directions (Amersham).

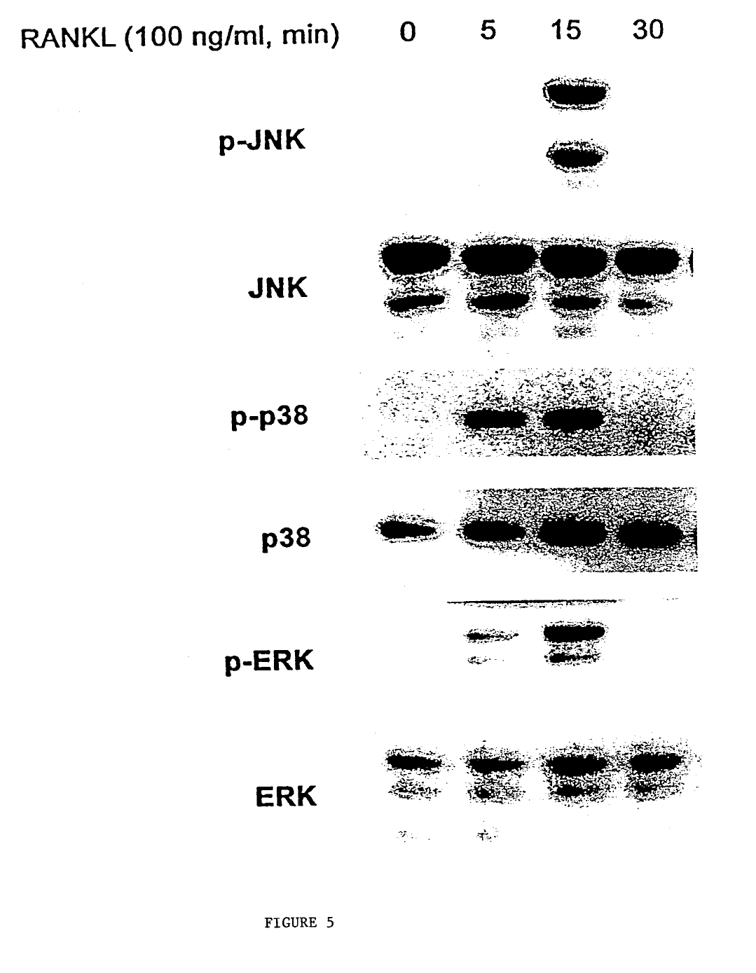

The results of the immunoblotting assay are depicted in FIG. 5. As can be seen from this figure, the total cellular amounts of JNK, p38, and ERK did not change significantly at any point of the assay. The phosphorylation (activation) of ERK and p38 was detected 5 minutes following the GST-RANKL stimulation, peaked at 10 minutes after RANK/GST-RANKL interaction, and was undetectable 30 minutes after the interaction. JNK was phosphorylated 15 minutes after the GST-RANKL stimulation, however the protein was also rapidly dephosphorylated so that by 30 minutes following GST-RANKL stimulation, phosphorylated forms of JNK were undetectable. The data indicated transient and short-lived activation of ERK, JNK, and p38 in murine osteoclast precursors following the GST-RANKL stimulation.

### Example 5

GST-RANKL rapidly activates Akt in murine osteoclast precursors. Osteoclast precursors were isolated, maintained, and manipulated as described in Example 1. Immunoblotting protocol was also the same as in Example 1, except that a primary antibody was specific for phospho-Akt, obtained from Cell Signaling.

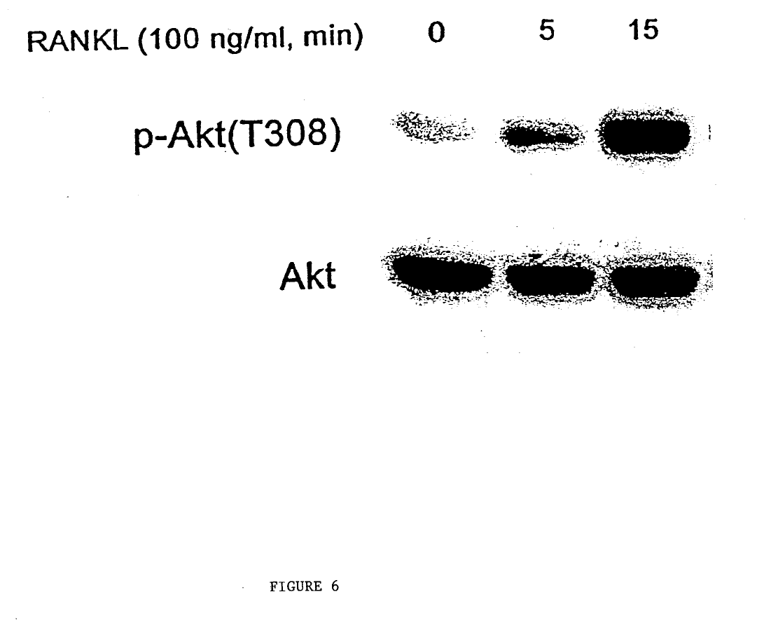

FIG. 6 shows that there was a detectable phosphorylation of Akt at the time of GST-RANKL stimulation, indicating rapid activation of this protein. Akt is a substrate for PI3 kinase, and in its active state is involved in anti-apoptotic signaling. Akt activation increased with time, i.e. the number of phosphorylated Akt molecules in osteoclast precursors increased with time. Thus, the activation of Akt was greater at 5 minutes than at 0 minutes, and it peaked at 15 minutes following GST-RANKL stimulation.

### Example 6

GST-RANKL-induced activation of MAP kinases is prolonged in murine osteoblasts. Primary osteoblasts were isolated from neonatal murine calvaria by sequential enzymatic digestion. Briefly, calvaria were minced and incubated at room temperature for 20 minutes with gentle shaking in an enzymatic solution containing 0.1% collagenase, 0.05% trypsin, and 4 mM NA₂EDTA in calcium- and magnesium-free phosphate buffered saline (PBS). This procedure was repeated to yield a total of six digests. The cells isolated from the last four to six digests were cultured in MEM containing 15% FBS, 50 μM ascorbic acid, and 10 mM β-glycerophosphate. Cells were maintained at 37° C. in a humidified atmosphere containing 6% C02, with daily replenishment of media and cytokines.

Following treatment with GST-RANKL at the indicated times and dosages, cells were lysed in RIPA buffer containing 10 mM Tris-HCl pH 7.4, 150 mM NaCl, 1% Nonidet P-40, 0.2% sodium deoxycholate, and 1 mM EDTA, with 1 mM Na₃PO₄, 1 mM NaF, and 1×protease inhibitor cocktail added immediately prior to use. Protein concentration was quantitated and standardized by Micro BCA Protein Assay (Pierce). Lysates were denatured by heat in Laemmli buffer, resolved by SDS-PAGE, and transferred onto nitrocellulose. Levels of total and phosphorylated ERK, JNK, p38, Akt, and IkBα were determined using primary and secondary antibodies according to the manufacturer's established protocols, with conventional chemiluminiscent detection. Membranes were stripped between hybridizations in PBS containing 10 μM β-mercaptoethanol and 2% SDS.

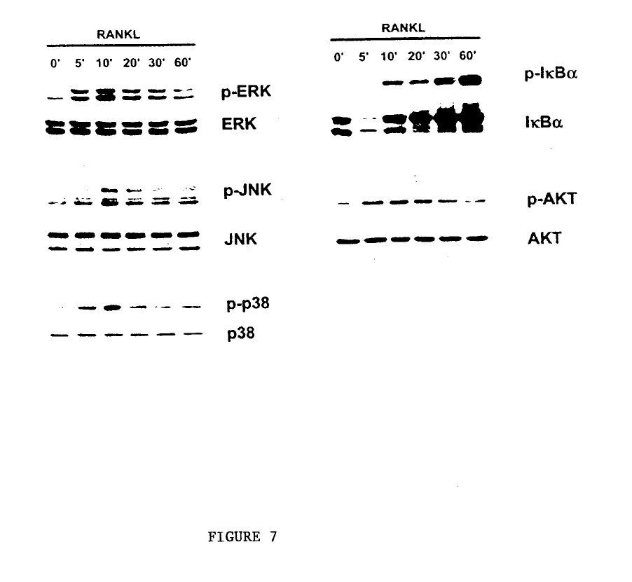

The results of the immunoblot assay measuring the activation of MAP kinases following GST-RANKL stimulation are shown in FIG. 7. GST-RANKL stimulation was performed as described in Example 5. The kinases whose phosphorylation was measured include ERK, JNK, p38, and Akt. Again, as seen in osteoclasts, the amount of total protein did not significantly change in the cell at any time points. However, all of the kinases tested exhibited prolonged activation in osteoblasts. Both ERKs were activated by 5 minutes after GST-RANKL stimulation, and their activation could be detected at 60 minutes following the stimulation. The activation of JNK, p38, and Akt was detectable at the time of GST-RANKL stimulation, and could be detected for at least 60 minutes following the stimulation. In addition, phosphorylation of IkBα was detected 10 minutes after the stimulation and it increased until the end of the assay (60 minutes), indicating increased translocation of NFkB into the nucleus. The data suggest that the pattern of MAP kinase activation is different from the activation of the same kinases in osteoclasts. The prolonged activation observed in osteoblasts seems to play a role in accelerated anabolic bone processes.

### Example 7

GST-RANKL-induced ERk1/2 activation is prolonged in murine osteoblast precursors. Osteoblast precursors were isolated and maintained according to the procedures set forth in Example 5. The immunoblotting was performed in the same manner as immunoblotting in Example 3.

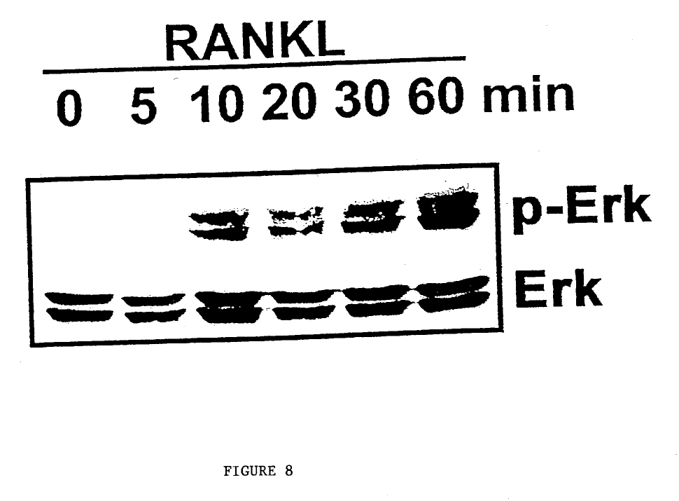

As observed in FIG. 8, ERK activation in osteoblast precursors was prolonged and it increased with time. Whereas in osteoblasts the activation was prolonged but did not change significantly over time, ERK activation in osteoblast precursors was first detected at 10 minutes following GST-RANKL stimulation, and it increased up to 60 minutes following the activation, which was the length of time for which the assay was performed.

### Example 8

In vivo stimulation of bone formation in mice results in 24% increase. Mice were anesthetized with a Ketamine/Xylazine cocktail (10 mg/kg ketamine and 10 mg/kg xylazine IP) and placed in left lateral recumbancy. The major trochanter and lateral femoral condyle of the right femur were identified and the intramedullary injection site was equidistant between these landmarks. Injections of 100 micrograms GST (control) or 100 micrograms GST-RANKL were made once a day for nine days with 29 gauge needles on tuberculin syringes.

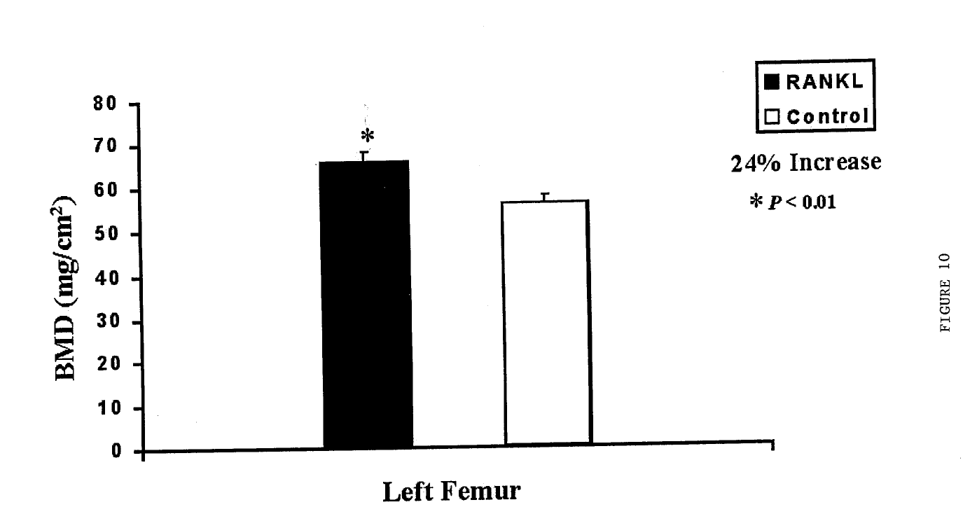

On day 9 the mice were re-anesthetized with Ketamine/Xylazine cocktail (10 mg/kg ketamine and 10 mg/kg xylazine IP) and dual energy x-ray absorptiometry (DEXA, Piximus) analysis was done on each animal. Plain radiographs were taken immediately following DEXA analysis (Faxitron, KV 0.15, time=20 sec). Animals were sacrificed by CO2 asphyxiation and both femurs harvested for histological analysis. The femurs were fixed in 10% buffered formalin for 48 hours and decalcified for 1 week. The results, showing a 24% increase in bone density of the femur of GST-RANKL treated mice versus the control, are shown in FIG. 10.

What is claimed is:

1. A method of screening osteogenic compounds, said method comprising the steps of:
(a) selecting a compound that activates some portion of the RANK pathway in an osteoblast or osteoblast precursor;
(b) performing a bone formation assay with said compound;
(c) determining the result of the bone formation assay, wherein a positive result marks said compound as possessing osteogenic potential.

2. The method of claim 1, further comprising identifying the compound that possesses osteogenic potential.

3. The method of claim 1, wherein the compound comprises a recombinant RANK ligand protein, a RANK ligand fusion protein, an analog, derivative or mimic thereof.

4. The method of claim 3, wherein the compound comprises the recombinant RANKL protein.

5. The method of claim 1, wherein said selecting comprises screening small molecule libraries for binding to RANK.

6. The method of claim 1, wherein said selecting comprises measuring the ability of a compound to directly bind RANK or to compete with a known binding partner of RANK including but not limited to RANKL or fragments, derivatives, or analogs therefrom.

7. The method of claim 1, wherein said selecting comprises modeling RANK-binding compounds by using a three-dimensional structural representation of a RANKL ectodomain crystal complex or a fragment thereof comprising elements involved in binding, and synthesizing such compounds.

8. The method of claim 1, wherein said selecting comprises screening phage display libraries for RANK-binding compounds.

9. A composition for enhancing bone formation comprising an effective amount of a compound screened by the method of claim 1.

10. A composition comprising an effective amount of the compound of claim 9 in a pharmaceutically acceptable carrier, adjuvant, solubilizer, stabilizer, and/or anti-oxidant.

11. A method of enhancing processes of bone formation comprising administering an effective amount of the composition of claim 9 to a subject in need of such enhancement.

12. The method of claim 11 further comprising intermittently administering an effective amount of the composition of claim 9 to a subject in need of such enhancement.

13. A method of screening osteogenic compounds, said method comprising the steps of:
(a) incubating a compound with osteoblasts or osteoblast precursors;
(b) measuring the activation of intracellular proteins in the RANK pathway in osteoblasts or osteoblast precursors indicative of bone formation, wherein the activation is determined by performing an activation assay specific for the intracellular protein in question.

14. The method of claim 13 further comprising identifying the compound whose activity resulted in the activation of the intracellular proteins.

15. The method of claim 13, wherein the intracellular proteins comprise kinases.

16. The method of claim 15, wherein the kinases comprise one or more of ERK1/2, PI3 kinase, IKK, Akt, JNK, and p38.

17. The method of claim 16, wherein the kinase comprises ERK1/2.

18. The method of claim 13, wherein the activation comprises phosphorylation of kinases.

19. The method of claim 18, wherein the kinases comprise one or more of ERK1/2, IKK, PI3 kinase, Akt, JNK, and p38.

20. The method of claim 17, wherein the activation comprises phosphorylation of ERK1/2.

21. The method of claim 13, wherein the activation of intracellular proteins is detected for at least about 30 minutes after the incubation step.

22. The method of claim 13, wherein the activation of intracellular proteins is detected for at least about 40 minutes.

23. The method of claim 13, wherein the activation of intracellular proteins is detected for at least about 60 minutes.

24. The method of claim 21, wherein the intracellular proteins are kinases.

25. The method of claim 24, wherein the kinase comprises ERK.

26. A method of screening and identifying osteogenic compounds, said method comprising the steps of:
(a) incubating a compound with osteoblasts or osteoblast precursors;
(b) measuring the activation of intracellular phosphatases in said osteoblasts or osteoblast precursors;
(c) performing a bone formation assay with the compound whose activity comprises inactivation of said phosphatases;
(d) determining the result of the bone formation assay, wherein a positive result identifies said compound as possessing osteogenic potential.

27. The method of claim 26, wherein the phosphatases are selected from the group consisting of MAPK specific phosphatases and Akt specific phosphatases.

28. The method of claim 26, wherein the phosphatases further comprise ERK1/2, NFKB, AKT, JNK, p38 or IKK specific phosphatases.

29. A method of screening osteogenic compounds, said method comprising the steps of:
(a) selecting a compound that binds to RANK;
(b) incubating the compound with osteoblasts or osteoblast precursors;
(c) measuring the activation of intracellular proteins in osteoblasts or osteoblast precursors indicative of bone formation, wherein the activation is determined by performing an activation assay specific for the intracellular protein in question.

30. The method of claim 29, wherein said selecting comprises measuring the ability of a compound to compete with RANKL for binding to RANK.

31. The method of claim 29, wherein said selecting comprises measuring the ability of a compound to compete with the surface loops of RANKL, or with a fragment, analog, or derivative therefrom.

32. The method of claim 29, wherein said selecting comprises measuring the ability of a compound to directly bind RANK.

33. The method of claim 21 further comprising identifying the compound whose activity resulted in the activation of the intracellular proteins.

34. The method of claim 29, wherein the intracellular proteins comprise kinases.

35. The method of claim 34, wherein the kinases comprise ERK1/2, PI3 kinase, IKK, Akt, JNK, and p38.

36. The method of claim 34, wherein the kinase comprises ERK.

37. The method of claim 29, wherein the activation comprises phosphorylation of kinases.

38. The method of claim 37, wherein the activation comprises phosphorylation of ERK1/2, IKK, PI3 kinase, Akt, JNK, and p38.

39. The method of claim 37, wherein the activation comprises phosphorylation of ERK1/2.

40. The method of claim 29, wherein the activation of intracellular proteins is detected for at least about 30 minutes.

41. The method of claim 29, wherein the activation of intracellular proteins is detected for at least about 40 minutes.

42. The method of claim 29, wherein the activation of intracellular proteins is detected for at least about 60 minutes.

43. The method of claim 40, wherein the intracellular proteins are kinases.

44. The method of claim 39, wherein the kinase comprises ERK.

45. A method of screening and identifying osteogenic compounds, said method comprising the steps of:
(a) selecting a compound that binds RANK;
(b) incubating the compound with osteoblasts, or osteoblast precursors, or related cells or cell lines;
(c) measuring the activation of intracellular phosphatases in said osteoblasts, osteoblast precursors, or related cells or cell lines;
(d) performing a bone formation assay with the compound whose activity comprises inactivation of said phosphatases;
(e) determining the result of the bone formation assay, wherein a positive result identifies said compound as possessing osteogenic potential.

46. The method of claim 45, wherein said selecting comprises measuring the ability of a compound to compete with RANKL for binding to RANK.

47. The method of claim 45, wherein said selecting comprises measuring the ability of a compound to compete with the contact surface loops of RANKL, or with a fragment, analog, or derivative therefrom.

48. The method of claim 45, wherein said selecting comprises measuring the ability of a compound to directly bind RANK.

49. The method of claim 45, wherein the phosphatases comprise MAPK specific phosphatases and Akt specific phosphatases.

50. The method of claim 45, wherein the phosphatases comprise ERK1/2, NFKB, AKT, JNK, p38 or IKK specific phosphatases.

51. A composition for enhancing bone formation comprising an effective amount of at least one compound identified by the method of claim 13.

52. A pharmaceutical composition comprising an effective amount of the compound of claim 51 in a pharmaceutically acceptable carrier, adjuvant, solubilizer, stabilizer, and/or anit-oxidant.

53. A composition for enhancing bone formation comprising an effective amount of at least one compound identified by the method of claim 26.

54. A pharmaceutical composition comprising an effective amount of the compound of claim 53 in a pharmaceutically acceptable carrier, adjuvant, solubilizer, stabilizer, and/or anti-oxidant.

55. A composition for enhancing bone formation comprising an effective amount of at least one compound identified by the method of claim 29.

56. A pharmaceutical composition comprising an effective amount of the compound of claim 54 in a pharmaceutically acceptable carrier, adjuvant, solubilizer, stabilizer, and/or anti-oxidant.

57. A composition for enhancing bone formation comprising an effective amount of at least one compound identified by the method of claim 44.

58. A pharmaceutical composition comprising an effective amount of the compound of claim 57 in a pharmaceutically acceptable carrier, adjuvant, solubilizer, stabilizer, and/or anti-oxidant.

59. A method of enhancing processes of bone formation comprising administering, in combination, effective amounts of at least two of the compositions selected from the compositions of claims 9, 10, 51, 52, 53, 54, 55, 56, 57 and 58.

60. A method of enhancing processes of bone formation comprising intermittently administering, in combination, effective amounts of at least two of the compositions selected from the compositions of claims 9, 10, 51, 52, 53, 54, 55, 56, 57 and 58.

61. A composition for enhancing bone formation comprising an effective amount of a compound that directly binds RANK on an osteoblast.

62. A composition for enhancing bone formation comprising an effective amount of a compound that directly activates at least a portion of the RANK pathway on an osteoblast.

63. A composition for enhancing bone formation comprising an effective amount of a compound that directly activates a protein in the RANK pathway of an osteoblast downstream of RANK.

64. The composition of claim 63 wherein the protein in the RANK pathway is selected from the group comprising: proto oncogene proteins, C-Fos, intracellular kinases, ERK 1/2, IKK, PI3 kinase, AkT, JNK and p38.

65. A method of identifying an osteogenic RANKL comprising:
(a) introducing a test RANK ligand into a culture of osteoblasts or osteoblast precursors;
(b) determining intensity of the response of the culture to the test RANK ligand at a time point that is at least fifteen minutes after the introduction of the candidate RANK ligand; and
(c) comparing the intensity of the response to the response of a known osteogenic RANKL.

66. The method of claim 65 wherein the time point of step (b) is at least about 40 minutes.

67. The method of claim 65 wherein the time point of step (b) is at least about 60 minutes.

68. A method of identifying an osteogenic RANKL comprising:
(a) introducing a candidate RANK ligand into replicate cultures of osteoblasts or osteoblast precursors;
(b) determining persistence of the response of the culture to the candidate RANK ligand by measuring the response at a first time point that is shortly after the introduction of the candidate RANK ligand and at a second time point that is up to one hour or longer after the introduction of the candidate RANK ligand; and
(c) comparing the persistence of the response to the response of a known osteogenic RANKL.
